(19) United States
(12) Patent Application Publication
(10) Pub. No.: US 2004/0211432 A1
(43) Pub. Date: Oct. 28, 2004

(54) Sensory disorder rotating platform and method

(76) Inventor: Gerald Toerner, Upland, CA (US)

Correspondence Address: PRAVEL INTELLECTUAL PROPERTY LAW, PC, 200 DAINGERFIELD RD, SUITE 400, ALEXANDRIA, VA, 22314, US

(21) Appl. No.: 10/807,468

(22) Filed: Mar. 24, 2004

Related U.S. Application Data
(60) Provisional application No. 60/453,730, filed on Apr. 1, 2003.

(51) Int. Cl. A61B 19/00

(52) U.S. Cl. 128/897

(57) ABSTRACT

A method and device for treating patients with sensory integration disorder is provided which comprises the steps of providing a platform that is rotatably connected to a base; placing a patient on the rotating platform in a first specified position; spinning the patient in one direction at a desired rotational speed; then spinning the patient in the opposite direction at a desired rotational speed. The method can include placing the patient in several different specified positions followed by spinning first in a clockwise and then in a counterclockwise direction. The method is repeated as required by the patient, based on the recommendations of a qualified occupational therapist.

### CROSS REFERENCE TO RELATED APPLICATIONS

### 37 C.F.R. § 1.77(b)(2)

This application claims priority from the following application:

U.S. Provisional Application No. 60/453,730, filed Apr. 1, 2003.

### BACKGROUND OF THE INVENTION

### 37 C.F.R. § 1.77(b)(5)

Sensory integration is defined as the neurological process of organizing the information we get from our bodies and from the world around us for use in daily life. Sensory integration occurs in the central nervous system and the main task of the central nervous system is to integrate the senses. According to Dr. Jean A. Ayres, over 80 percent of the nervous system is involved in processing or organizing sensory input, and thus the brain is primarily a sensory processing machine.

Sensory integration generally involves modulation, inhibition, habituation and facilitation. Modulation is the term used to describe the brain's regulation of its own activity and therefore, of our activity level. Modulation balances the flow of sensory information coming into the central nervous system. The brain turns on, or turns off, the neural switches of all the sensory systems, so that they work in tandem to keep us in sync.

Inhibition in this context is defined as the neurological process that reduces connections between sensory intake and behavioral output. Inhibition allows us to ignore sensations that occur in an appropriate manner.

Habituation is a process defined as tuning out a familiar message after we have become accustomed to it, as appropriate.

Facilitation is the neurological process that promotes connections between sensory intake and behavioral output. When inhibition and facilitation are balanced, we can make smooth transitions from one state to another, where a “state” refers to a degree of attentiveness, mood or motor (movement) response.

No part of the central nervous system works alone. Messages must go back and forth from one part to another, so that touch can aid vision, vision can aid balance, balance can aid body awareness, body awareness can aid movement and movement can aid learning. When sensory messages come in, and motor messages go out, in a synchronized way, we can do what we need to do.

Sensory Integration Disorder is defined as the brain's inability to process sensations efficiently. Having Sensory Integration Disorder does not imply that there is damage to the brain, but instead according to Dr. Ayres, more of a situation where there is a “traffic jam in the brain.” Sensory Integration Disorder occurs when the open-ended, reciprocal process of intake/organization/output is disrupted.

The vestibular system tells us where our heads and bodies are in relation to the surface of the earth. The vestibular system takes in sensory messages about balance and movement from the neck, eyes, and body; sends those messages to the central nervous system for processing; and then helps to generate muscle tone that allows us to move smoothly and efficiently. The vestibular system tells us whether we are moving or standing still, and whether objects are moving or motionless in relation to our body. It also informs us of what direction we are going in, and how fast we are going.

The receptors for vestibular sensations are in the inner ear, a “vestibule” through which sensory messages pass. Movement and gravity stimulate the receptors. Dr. Ayres has defined the vestibular system as the unifying system. It forms the basic relationship of a person to gravity and to the physical world. All other types of sensation are processed in reference to the basic vestibular information. The activity in the vestibular system provides a “framework” for the other aspects of our experience. Vestibular input seems to “prime” the entire nervous system to function effectively. When the vestibular system does not function in a consistent and accurate way, the interpretation of other sensations will be inconsistent and inaccurate, and the nervous system will have trouble “getting started.”

Platform swings and hammocks are currently being used to treat sensory integration disorder, but unfortunately, they do not provide for consistent input to the child. What is needed is a system and method to assist children to improve the processing in the brain of sensations perceived through the inner ear of their vestibular system.

### BRIEF SUMMARY OF THE INVENTION

### 37 C.F.R. § 1.77(b)(6)

The invention relates to a method and device for treating patients with sensory integration disorder comprising the steps of providing a platform that is rotatably connected to a base; placing a patient on the platform in a first specified position; spinning the patient on the platform in one direction at a desired rotational speed; then spinning the patient on the platform in the opposite direction at a desired rotational speed.

The treatment method may also include the additional step of placing the patient on the platform in a second specified position; spinning the platform in one direction at a desired rotational speed; then spinning the platform in the opposite direction at a desired rotational speed.

The treatment method may also include the additional step of placing the patient on the platform in a third specified position; spinning the platform in one direction at a desired rotational speed; then spinning the platform in the opposite direction at a desired rotational speed.

The desired rotational speed is approximately 30 revolutions per minute. The steps of the method are typically performed sequentially at intervals of 72 hours maximum.

The platform generally includes straps for the patient to hold onto that are located at positions of approximately 10:00 O'clock and 2:00 O'clock. A bearing assembly, or other rotational mechanism is positioned between the platform and the base to allow the platform to rotate freely relative to the base. The top of the platform may be covered with a non-skid material so that the patient does not slide about the surface of the platform.

The three specified positions in which the patient is placed include, on the patient's left side, the patient's right side and the patient's posterior. During the treatment method, a different position selected from the three different positions so that a different position is selected for each of the three different positions.

The inventor has found that the treatment method keeps the patient's stress level and behavior at a more typical level, thereby allowing the patient to be more like their peers. Moreover, after treatment the patient feels more organized internally which allows them to feel calm and it reduces the stress that can create unwanted behavior.

The treatment method allows for a more consistent input than the platform swings and hammocks that are currently being used by most therapists. The current treatment method and platform are also easier to provide in a private home, thereby relieving the patient from having to attend a therapy session outside of the home. The method and device should only be used according to the instruction and recommendation of a qualified occupational therapist.

### BRIEF DESCRIPTION OF THE DRAWINGS

### 37 C.F.R. § 1.77(b)(7)

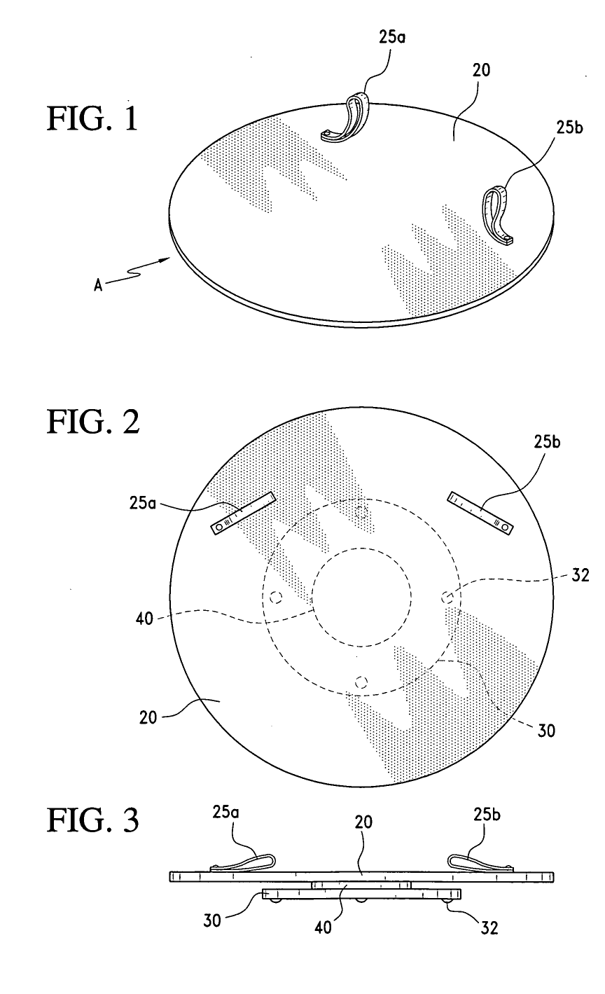

FIG. 1 shows an isometric view of the rotating disc used with the treatment method.

FIG. 2 shows a top view of the rotating disc of FIG. 1.

FIG. 3 shows an end view of the rotating disc of FIG. 1.

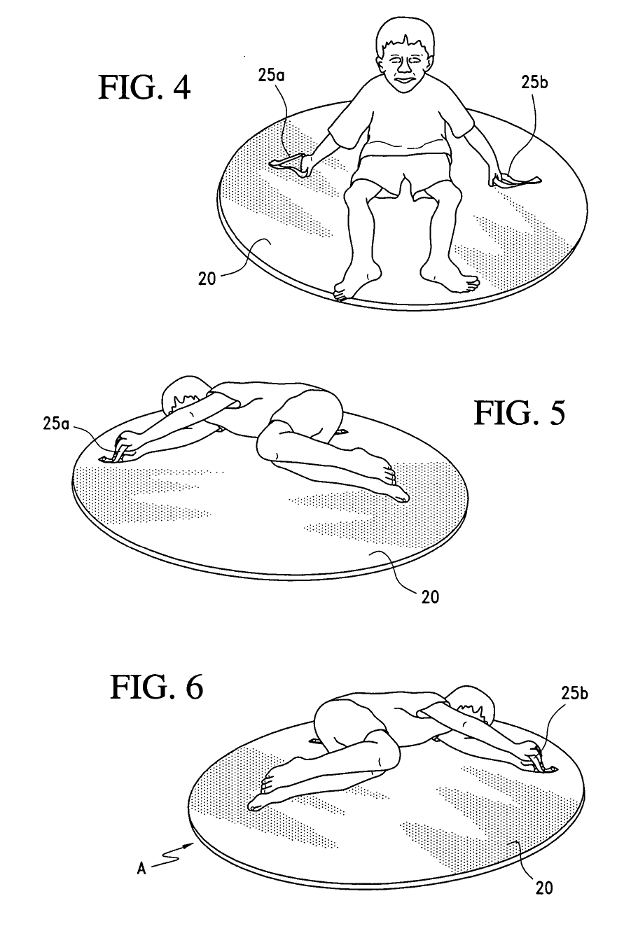

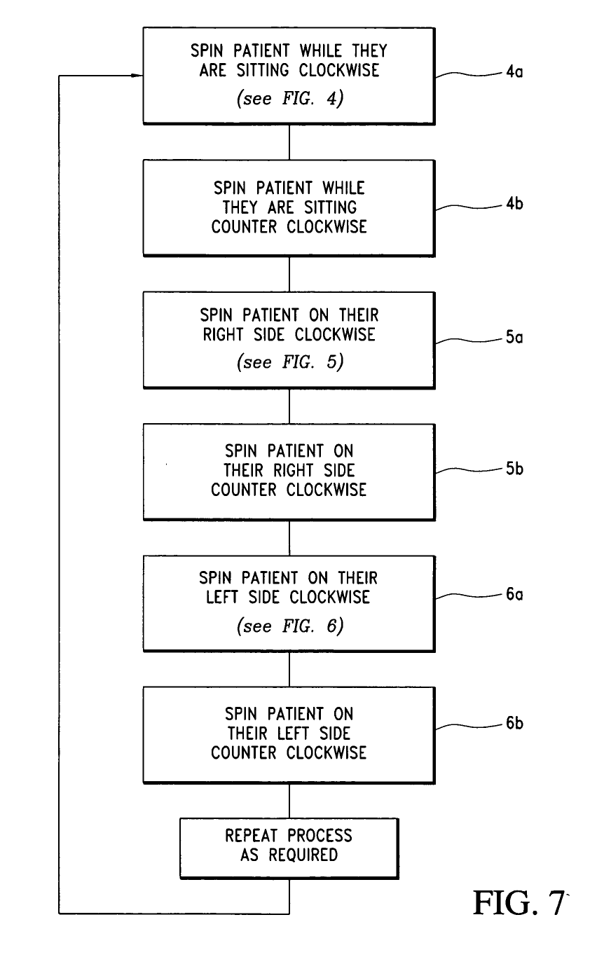

FIG. 4 shows an isometric view of the rotating disc of FIG. 1 with a patient sitting in the first of three specified positions during the treatment method.

FIG. 5 shows an isometric view of the rotating disc of FIG. 1 with a patient lying in the second of three specified positions during the treatment method.

FIG. 6 shows an isometric view of the rotating disc of FIG. 1 with a patient lying in the third of three specified positions during the treatment method.

FIG. 7 shows a flowchart of the treatment method.

### DETAILED DESCRIPTION OF THE INVENTION

### 37 C.F.R. § 1.77(b)(8)

The rotating platform assembly used with the inventive method is shown in FIGS. 1-3 and is identified generally as A. The platform 20 is constructed of plywood or other structural material. It includes handles 25a and 25b positioned such that a patient can conveniently grasp each handle. The left handle 25a is positioned at approximately a 10:00 O'clock position and the right handle is positioned at approximately a 2:00 O'clock position. The surface of the platform 20 may be covered with a non-skid material so that the patient does not slide on the platform 20.

The platform 20 is positioned on a base 30. A bearing assembly 40 is positioned between the platform 20 and the base 30, which allows the platform 20 to rotate freely about the base 30. The bearing assembly 40 illustrated in the preferred embodiment is 12 inches in diameter but other size bearing assemblies may also be used.

Feet 32 typically are uniformly positioned about the base 30. The use of feet 32 on the base 30 prevents the base 30 from scarring up the underlying floor on which the rotating platform assembly A is located and also prevents the base 30 itself from rotating or sliding relative to the underlying floor. The feet 32 are constructed of rubber or other polymer.

The three (3) specified positions for the patient are illustrated in FIGS. 4-6. In FIG. 4, the patient is seated; in FIG. 5, he or she is on their right side and in FIG. 6 he or she is on their left side. When the patient is on his or her left side (FIG. 6), they will typically hold onto the right handle 25b to stay secure on the rotating platform assembly A. Similarly, when the patient is on his or her right side (FIG. 5), they will typically hold onto the left handle 25b. When the patient is in the seated position, they hold onto both the left handle 25a and to the right handle 25b.

The treatment method is illustrated in the flow chart in FIG. 7. The patient is first spun clockwise at a desired rotational speed while he or she is seated (4a). While the patient is still seated (4b) he or she is spun on the rotating platform assembly A counter clockwise. The patient then changes position to his or her right side (5a) and he is spun at a desired rotational speed clockwise. While the patient is still positioned on his or her right side (5b) he or she is spun on the rotating platform assembly A counter clockwise. The patient then changes position to his or her left side (6a) and he is spun at a desired rotational speed clockwise. While the patient is still on his or her left side (6b) he or she is spun on the rotating platform assembly A counter clockwise.

Following the steps 4a, 4b, 5a, 5b, 6a and 6b a period of approximately 72 hours is allowed to pass before repeating the method steps again. The typical spinning speed is approximately 30 revolutions per minute.

It has been found that after treatment, the patient feels more organized internally, which allows them to be calm and it reduces the stresses that create unwanted behavior. The rotating platform assembly A allows a more consistent input and as a result, is more effective than the platform swings and hammocks that are currently being used by most therapists. It is also much easier to keep in a private home, which relieves the patient from having to travel to a therapy session when required.

The method is only to be used under the direction and recommendations of a qualified occupational therapist.

The foregoing disclosure and description of the invention are illustrative and explanatory thereof, and various changes in the speed of rotation, size of components, order of steps, order of patient positions, as well as changes in the details of the illustrated embodiments may be made without departing from the spirit or scope of the invention.

1. A method for treating patients with sensory integration disorder comprising:
a. providing a platform that is rotatably connected to a base;
b. placing a patient on said platform in a first specified position;
c. spinning the patient on said platform in one direction at a desired rotational speed;
d. spinning the patient on said platform in the opposite direction at a desired rotational speed.

2. A treatment method according to claim 1 comprising the following additional steps:
a. placing the patient on said platform in a second specified position;
b. spinning the patient on said platform in one direction at a desired rotational speed;
c. spinning the patient on said platform in the opposite direction at a desired rotational speed.

3. A treatment method according to claim 2 comprising the following additional steps:
a. placing the patient on said platform in a third specified position;
b. spinning the patient on said platform in one direction at a desired rotational speed;
c. spinning the patient on said platform in the opposition direction at a desired rotational speed.

4. A treatment method according to claim 3 wherein the desired rotational speed is approximately 30 revolutions per minute.

5. A treatment method according to claim 3 wherein the steps are performed sequentially at intervals of approximately 72 hours thereby creating calm to the patient and reducing stresses that create unwanted behavior.

6. A treatment method according to claim 1 comprising the additional step of providing a plurality of handles on said platform to which the patient can hold onto during said treatment steps.

7. A treatment method according to claim 6 wherein said handles are positioned at the locations of approximately 10:00 O'clock and 2:00 O'clock.

8. A treatment method according to claim 1 wherein a bearing assembly is positioned between said platform and said base to allow said platform to rotate relative to said base.

9. A treatment method according to claim 1 wherein said first specified position is selected from the group consisting essentially of: the patient's left side, the patient's right side, and the patient's posterior.

10. A treatment method according to claim 2 wherein said second specified position is different from the first specified position and is selected from the group consisting essentially of: the patient's left side, the patient's right side, and the patient's posterior.

11. A treatment method according to claim 3 wherein said third specified position is different from the first or second specified positions and is selected from the group consisting essentially of: the patient's left side, the patient's right side, and the patient's posterior.

12. A treatment method according to claim 1 wherein said platform is covered in a non-skid material.

13. A device used with a method of treating sensory disorder wherein the method includes the steps of having a patient sit on a rotating device; spinning the patient in a clockwise and a counterclockwise direction; then the patient lies down on one side, and spins in a clockwise and a counterclockwise direction; then the patient lies down on their other side, and spins in a clockwise and a counterclockwise direction; the device comprising:
a. a disc member rotatably attached to a base member;
b. a bearing assembly positioned between said disc member and said base member to allow said disc member to rotate freely about said base member; and
c. a plurality of handles on said disc member for a patient to hold onto when said disc member is spinning.

14. A device as claimed in claim 13 further comprising a non-skid material on the surface of said disc member for preventing the patient from slipping on said disc member.

15. A device as claimed in claim 13 further comprising feet about the lower surface of said base for preventing said base from rotating relative to the underlying surface on which the base is placed.

16. A device as claimed in claim 15 wherein said feet are constructed of a polymer material.

17. A device as claimed in claim 16 wherein said feet are constructed of rubber.
